SEQUENCE LISTING (4 sequences)

SEQ ID NO: 1 - protein, 320 aa, Homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 2 - protein, 320 aa, Artificial
  Synthetic Mutant2 R227A annexin A5
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDAETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 3 - protein, 320 aa, Artificial
  Synthetic Mutant3 E228A annexin A5
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRATSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 4 - protein, 320 aa, Artificial
  Synthetic Mutant5 Y257A annexin A5
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYAAMKGAGTDDHTLIRVMVSRSEIDLFNIRKEFRKNFATSLYSMIKGDTSGDYKKALLLLCGEDD

(19) United States
(12) Patent Application Publication
(10) Pub. No.: US 2024/0141001 A1
(43) Pub. Date: May 2, 2024

(54) MUTATED ANNEXIN A5 POLYPEPTIDES AND USES THEREOF FOR THERAPEUTIC PURPOSES

(71) Applicants: INSTITUT NATIONAL DE LA SANTÉ ET DE LA RECHERCHE MÉDICALE, Paris (FR); SORBONNE UNIVERSITÉ, Paris (FR); UNIVERSITÉ PARIS CITÉ, Paris (FR)

(72) Inventors: Olivier BLANC-BRUDE, Paris (FR); Lubka ROUMENINA, Paris (FR)

(21) Appl. No.: 18/569,283

(22) PCT Filed: Jun. 14, 2022
(86) PCT No.: PCT/EP2022/066102

§ 371 Date: Dec. 12, 2023

(30) Foreign Application Priority Data
Jun. 14, 2021 (EP) 21305810.0

(51) Int. Cl. C07K 14/47; A61P 7/06; A61P 31/14

(52) U.S. Cl. CPC C07K 14/47; A61P 7/06; A61P 31/14; A61K 38/00

(57) ABSTRACT

The inventors generated new mutated Annexin A5 polypeptides to which the binding of heme is drastically reduced, but the binding to phosphatidylserine-bearing membranes remains at the same level in presence of heme or during intravascular hemolysis. Thus mutated polypeptides can therefore be particularly suitable for therapeutic purposes, in order to harness reactions enhanced by PS or PS-bearing membranes, most particularly in pathologies where thrombotic, vaso-occlusive and hemolytic conditions co-exist.

### FIELD OF THE INVENTION

The present invention is in the field of medicine, in particular haematology.

### BACKGROUND OF THE INVENTION

Annexin A5 belongs to the Annexin family which is a group of proteins which are highly conserved in evolution. They are structurally homologous, and are considered to be membrane-bound elements regulated by Ca²⁺. Annexin A5 has high affinity for phosphatidylserine (PS) that is exposed to the outer plasma membrane leaflet. Beyond that, Annexin A5 can bind to any PS-containing phospholipid bilayer of almost all tiny forms of membranous vesicles like blood platelets, exosomes, or even nanostructured liposomes. It is known that Annexin A5 has a wide range of utilities in medicine, in providing direct therapeutic effects. Examples include the use of Annexin A5 for preventing a vaso-occlusive crisis in a patient afflicted with sickle cell disease as described in WO2012120130, for prevention of atherothrombosis and/or plaque rupture as described in WO2005099744; for the treatment of vascular dysfunction, reducing ischemic pain and/or treatment of a vascular disease rupture as described in WO2009077764; for the prophylaxis or treatment of restenosis as described in WO 2009103977; for use in inhibiting the activity of oxidized cardiolipin (oxCL) and for treating, preventing and/or reducing the risk of developing a cardiovascular disease, an auto-immune disease or inflammatory condition as described in WO2010/069605; and for the prevention and/or reduction of peri- or postoperative complications following surgical intervention, such as complications following vascular surgery, especially peripheral vascular surgery as described in WO2012136819. As such, Annexin A5 represents a protein of high therapeutic interest and potential. It was recently shown that heme binds to Annexin A5 during hemolysis and prevents its interaction with the cell membrane phosphatidylserine. These results therefore suggest that the therapeutic effects of Annexin A5 can be reduced in hemolytic conditions.

### SUMMARY OF THE INVENTION

The present invention is defined by the claims. In particular, the present invention relates to mutated Annexin A5 polypeptides and uses thereof for therapeutic purposes.

### DETAILED DESCRIPTION OF THE INVENTION

### Main Definitions

As used herein, the terms “polypeptide”, “peptide”, and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

As used herein, the expression “derived from” refers to a process whereby a first component (e.g., a first polypeptide), or information from that first component, is used to isolate, derive or make a different second component (e.g., a second polypeptide that is different from the first one).

As used herein, the term “Annexin A5” has its general meaning in the art and refers to a polypeptide that belongs to the Annexin family and that binds to externalized phosphatidylserine in a calcium dependent manner. The term is also known as Anchorin CII, Annexin V, Annexin-5n Calphobindin I (CBP-I), Endonexin II, Lipocortin V, Placental anticoagulant protein 4 (PP4), Placental anticoagulant protein I (PAP-I), Thromboplastin inhibitor and Vascular anticoagulant-alpha (VAC-alpha). An exemplary amino acid sequence for Annexin A5 is shown as SEQ ID NO:1.

>NP_001145.1 Annexin A5 [Homo sapiens]
SEQ ID NO: 1
MAQVLRGTVTDFPGFDERADAETLRKAMKGLGTDEESILTLLTSRSNAQ
RQEISAAFKTLFGRDLLDDLKSELTGKFEKLIVALMKPSRLYDAYELKH
ALKGAGTNEKVLTEIIASRTPEELRAIKQVYEEEYGSSLEDDVVGDTSG
YYQRMLVVLLQANRDPDAGIDEAQVEQDAQALFQAGELKWGTDEEKFIT
IFGTRSVSHLRKVFDKYMTISGFQIEETIDRETSGNLEQLLLAVVKSIR
SIPAYLAETLYYAMKGAGTDDHTLIRVMVSRSEIDLFNIRKEFRKNFAT
SLYSMIKGDTSGDYKKALLLLCGEDD

As used herein, the “percent identity” between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the Needleman and Wunsch algorithm (Needleman, Saul B. & Wunsch, Christian D. (1970). “A general method applicable to the search for similarities in the amino acid sequence of two proteins”. Journal of Molecular Biology. 48 (3): 443-53.). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification. According to the invention a first amino acid sequence having at least 90% of identity with a second amino acid sequence means that the first sequence has 90; 91; 92; 93; 94; 95; 96; 97; 98; 99 or 100% of identity with the second amino acid sequence.

As used herein, the term “mutation” has its general meaning in the art and refers to a substitution, deletion or insertion. In particular, the term “substitution” means that a specific amino acid residue at a specific position is removed and another amino acid residue is inserted into the same position. Within the specification, the mutation are references according to the standard mutation nomenclature.

As used herein, the term “phosphatidylserine” or “PS” has its general meaning in the art and refers to a phospholipid that is a component of the cell membrane. More particularly, PS is a phospholipid-more specifically a glycerophospholipid-which consists of two fatty acids attached in ester linkage to the first and second carbon of glycerol and serine attached through a phosphodiester linkage to the third carbon of the glycerol.

As used herein, the term “heme” refers to a coordination compound of porphyrin (or derivative thereof) and mainly bivalent or trivalent iron, and it is also called iron porphyrin and hematin. In the present invention no particular restriction is placed on the heme that is used and a natural heme, for example, heme a, heme b (protoheme IX), heme c, variant heme c, heme d, heme dl, siroheme (Sirohaem), and heme o can be used (see A. Messerschmidt, R. Huber, T. Poulos, and K. Wieghardt (Eds), Handbook of Metalloproteins Vol. 1, John Wiley & Sons, New York, 2001, etc.).

As used herein, the term “affinity”, as used herein, means the strength of the binding of polypeptide (e.g. Annexin A5 polypeptide) to a particular ligand. The affinity of polypeptide is given by the dissociation constant Kd. The affinity constant Ka is defined by 1/Kd. Methods for measuring the K_{D} of a polypeptide are well known in the art and include, without limitation, surface plasmon resonance (SPR) technology in a BIAcore 3000 instrument. BIACORE® (GE Healthcare, Piscaataway, NJ) is one of a variety of surface plasmon resonance assay formats that are routinely used to determine the affinity. Affinities of antibodies can be readily determined using other conventional techniques, for example, those described by Scatchard et al., (Ann. N.Y. Acad. Sci. USA 51:660 (1949)).

As used herein, the term “hemolysis” refers to the destruction or dissolution of red blood cells, with release of hemoglobin (as free hemoglobin) and heme.

As used herein, the term “hemolytic anemia” as used herein refers to any condition in which the number of erythrocytes (red blood cells) per mm or the amount of hemoglobin in 100 ml of blood is less than normal resulting from the destruction of erythrocytes.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the expression “therapeutically effective amount” is meant a sufficient amount of the active ingredient of the present invention to induce an immune response at a reasonable benefit/risk ratio applicable to the medical treatment.

As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical-Sciences, Sixteenth Edition, E. W. Martin (Mack Publishing Co., Easton, Pa., 1980) discloses various carriers used in formulating pharmaceutical compositions and known techniques for the preparation thereof.

As used herein, the term “polypeptide that comprises an amino acid sequence” or “polypeptide comprising an amino acid sequence” refer to both polypeptide comprising or consisting of said amino acid sequence.

Mutated Annexin A5 Polypeptides of the Present Invention:

The first object of the present invention relates to a mutated Annexin A5 polypeptide that comprises an amino acid sequence having at least 90% of identity with the amino acid sequence as set forth in SEQ ID NO: 1 wherein at least one amino acid at position 227, 228 or 257 is mutated.

In some embodiments, the amino acid at position 227, 228 or 257 in SEQ ID NO:1 is substituted.

In some embodiments, the amino acids at positions 227, 228 and 257 in SEQ ID NO:1 are substituted.

In some embodiments, the mutated Annexin A5 polypeptide of the invention does not comprises an amino acid sequence as set forth in SEQ ID NO: 1 wherein the amino acid (E) at position 72 is substituted by the amino acid (D), the amino acid (D) at position 144 is substituted by the amino acid (N), the amino acid (E) at position 228 is substituted by the amino acid (A) and the amino acid (D) at position 303 is substituted by the amino acid (N) (AA5 mutant M1234 as disclosed in Kenis et al. J Nucl Med 2010 and Kenis et al. JBC Papers 2004).

In some embodiments, the mutated Annexin A5 polypeptide of the invention does not comprises an amino acid sequence as set forth in SEQ ID NO: 1 wherein the amino acid (D) at position 144 is substituted by the amino acid (N) and the amino acid (E) at position 228 is substituted by the amino acid (A) (AA5 mutant M23 as disclosed in Kenis et al. J Nucl Med 2010 and Kenis et al. JBC Papers 2004).

In some embodiments, the amino acid (R) at position 227 of SEQ ID NO:1 is substituted by the amino acid (A) (rhA5-‘R’).

In some embodiments, the amino acid (E) at position 228 of SEQ ID NO:1 is substituted by the amino acid (A) (rhA5-‘E’).

In some embodiments, the amino acid (Y) at position 257 of SEQ ID NO:1 is substituted by the amino acid (A) (rhA5-‘Y’).

Therefore in some embodiments, the mutated Annexin A5 polypeptide of the present invention comprises an amino acid sequence having at least 90% of identity with the amino acid sequence as set forth in SEQ ID NO: 1 that comprises at least one mutation selected from the group consisting of R227A, E228A and Y257A.

In some embodiments, the mutated Annexin A5 polypeptide of the present invention comprises an amino acid sequence having at least 90% of identity with the amino acid sequence as set forth in SEQ ID NO: 2, 3 or 4.

>NP_001145.1.2 Mutant2 R227A annexin A5 [Homo sapiens]
SEQ ID NO: 2
MAQVLRGTVTDFPGFDERADAETLRKAMKGLGTDEESILTLLTSRSNAQRQEISAAFKTLFGRDLLDDLK
SELTGKFEKLIVALMKPSRLYDAYELKHALKGAGTNEKVLTEIIASRTPEELRAIKQVYEEEYGSSLEDD
VVGDTSGYYQRMLVVLLQANRDPDAGIDEAQVEQDAQALFQAGELKWGTDEEKFITIFGTRSVSHLRKVF
DKYMTISGFQIEETIDAETSGNLEQLLLAVVKSIRSIPAYLAETLYYAMKGAGTDDHTLIRVMVSRSEID
LFNIRKEFRKNFATSLYSMIKGDTSGDYKKALLLLCGEDD
>NP_001145.1.3 Mutant3 E228A annexin A5 [Homo sapiens]
SEQ ID NO: 3
MAQVLRGTVTDFPGFDERADAETLRKAMKGLGTDEESILTLLTSRSNAQRQEISAAFKTLFGRDLLDDLK
SELTGKFEKLIVALMKPSRLYDAYELKHALKGAGTNEKVLTEIIASRTPEELRAIKQVYEEEYGSSLEDD
VVGDTSGYYQRMLVVLLOANRDPDAGIDEAQVEQDAQALFQAGELKWGTDEEKFITIFGTRSVSHLRKVF
DKYMTISGFQIEETIDRATSGNLEQLLLAVVKSIRSIPAYLAETLYYAMKGAGTDDHTLIRVMVSRSEID
LFNIRKEFRKNFATSLYSMIKGDTSGDYKKALLLLCGEDD
>NP_001145.1.5 Mutant5 Y257A annexin A5 [Homo sapiens]
SEQ ID NO: 4
MAQVLRGTVTDFPGFDERADAETLRKAMKGLGTDEESILTLLTSRSNAQRQEISAAFKTLFGRDLLDDLK
SELTGKFEKLIVALMKPSRLYDAYELKHALKGAGTNEKVLTEIIASRTPEELRAIKQVYEEEYGSSLEDD
VVGDTSGYYQRMLVVLLOANRDPDAGIDEAQVEQDAQALFQAGELKWGTDEEKFITIFGTRSVSHLRKVF
DKYMTISGFQIEETIDRETSGNLEQLLLAVVKSIRSIPAYLAETLYAAMKGAGTDDHTLIRVMVSRSEID
LFNIRKEFRKNFATSLYSMIKGDTSGDYKKALLLLCGEDD

In some embodiments, the mutated Annexin A5 polypeptide of the present invention comprises the amino acid sequence as set forth in SEQ ID NO: 2, 3 or 4.

In some embodiments, the mutated Annexin A5 polypeptide of the present invention comprises the amino acid sequence as set forth in SEQ ID NO:3.

According to the present invention, the mutated Annexin A5 polypeptide keeps the same affinity for phosphatidylserine as for the non-mutated Annexin A5 polypeptide while having a reduced affinity for heme. Typically, the mutated Annexin A5 polypeptide of the present invention has a higher Kd value for Heme than the non-mutated Annexin A5 polypeptide. Typically the Kd value for Heme of the mutated Annexin A5 polypeptide of the present invention is 2; 2;5; 3; 3,5; 4; 4,5 times higher than the Kd value of the non-mutated Annexin A5 polypeptide. In some embodiments, the Kd value for PS of the mutated Annexin A5 polypeptide of the present invention is 2; 2;5; 3; 3,5; 4; 4,5 times lower than the Kd value of the non-mutated Annexin A5 polypeptide in presence of heme.

According to the invention, the mutated Annexin A5 polypeptides of the invention are produced by conventional automated peptide synthesis methods or by recombinant expression. General principles for designing and making proteins are well known to those of skill in the art. The mutated Annexin A5 polypeptides of the invention may be synthesized in solution or on a solid support in accordance with conventional techniques. Various automatic synthesizers are commercially available and can be used in accordance with known protocols as described in Stewart and Young; Tam et al., 1983; Merrifield, 1986 and Barany and Merrifield, Gross and Meienhofer, 1979. The mutated Annexin A5 polypeptides of the invention may also be synthesized by solid-phase technology employing an exemplary peptide synthesizer such as a Model 433A from Applied Biosystems Inc. The purity of any given protein; generated through automated peptide synthesis or through recombinant methods may be determined using reverse phase HPLC analysis. Chemical authenticity of each peptide may be established by any method well known to those of skill in the art. As an alternative to automated peptide synthesis, recombinant DNA technology may be employed wherein a nucleotide sequence which encodes a protein of choice is inserted into an expression vector, transformed or transfected into an appropriate host cell and cultivated under conditions suitable for expression as described herein below. Recombinant methods are especially preferred for producing longer polypeptides. A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors (Giga-Hama et al., 1999); insect cell systems infected with virus expression vectors (e.g., baculovirus, see Ghosh et al., 2002); plant cell systems transfected with virus expression vectors (e.g., cauliflower mosaic virus, CaMV; tobacco mosaic virus, TMV) or transformed with bacterial expression vectors (e.g., Ti or pBR322 plasmid; see e.g., Babe et al., 2000); or animal cell systems. Those of skill in the art are aware of various techniques for optimizing mammalian expression of proteins, see e.g., Kaufman, 2000; Colosimo et al., 2000. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below. Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

In some embodiments, the polypeptide of the invention is not fused to a growth factor.

In some embodiment, the polypeptide of the invention is not fused to Insulin-like Growth Factor One (IGF-1) or Neuregulin 1 (NRG).

In some embodiment, the polypeptide of the invention is not fused to Insulin-like Growth Factor One (IGF-1).

In some embodiment, the polypeptide of the invention is not fused to Insulin-like Growth Factor One (IGF-1), wherein the IGF-1 comprises an amino sequence set forth as SEQ ID NO:18, 19, 23, 24, 28, 29, or 120 described in US2020/0207826.

In some embodiments, it is contemplated that the mutated Annexin A5 polypeptides of the invention used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain. Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

A further object of the present invention relates to a polynucleotide that encodes for a mutated Annexin A5 polypeptide of the present invention.

In some embodiments, the polynucleotide of the present invention is included in a suitable vector, such as a plasmid, cosmid, episome, artificial chromosome, phage or a viral vector. So, a further object of the invention relates to a vector comprising a nucleic acid encoding for a mutated Annexin A5 polypeptide of the invention. Typically, the vector is a viral vector which is an adeno-associated virus (AAV), a retrovirus, bovine papilloma virus, an adenovirus vector, a lentiviral vector, a vaccinia virus, a polyoma virus, or an infective virus. In some embodiments, the vector is an AAV vector. As used herein, the term “AAV vector” means a vector derived from an adeno-associated virus serotype, including without limitation, AAV1, AAV2, AAV3, AAV4, AAV5, AAV6, AAV7, AAV8, AAV9, and mutated forms thereof. AAV vectors can have one or more of the AAV wild-type genes deleted in whole or part, preferably the rep and/or cap genes, but retain functional flanking ITR sequences. Retroviruses may be chosen as gene delivery vectors due to their ability to integrate their genes into the host genome, transferring a large amount of foreign genetic material, infecting a broad spectrum of species and cell types and for being packaged in special cell-lines. In order to construct a retroviral vector, a nucleic acid encoding a gene of interest is inserted into the viral genome in the place of certain viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV 1, HIV 2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are known in the art, see, e.g. U.S. Pat. Nos. 6,013,516 and 5,994,136, both of which are incorporated herein by reference. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest. Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene into that packaging cell yields a producer cell which releases infectious viral particles carrying the foreign gene of interest. The env preferably is an amphotropic envelope protein which allows transduction of cells of human and other species. Typically, the polynucleotide or the vector of the present invention include “control sequences'”, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell. Another nucleic acid sequence, is a “promoter” sequence, which is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

A further object of the present invention relates to a host cell transformed with the polynucleotide of the present invention. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”. In a particular embodiment, for expressing and producing the mutated Annexin A5 polypeptides of the present invention, prokaryotic cells, in particular E. coli cells, will be chosen. Actually, according to the invention, it is not mandatory to produce the mutated Annexin A5 polypeptides of the present invention in a eukaryotic context that will favour post-translational modifications (e.g. glycosylation). Furthermore, prokaryotic cells have the advantages to produce protein in large amounts. If a eukaryotic context is needed, yeasts (e.g. saccharomyces strains) may be particularly suitable since they allow production of large amounts of proteins. Otherwise, typical eukaryotic cell lines such as CHO, BHK-21, COS-7, C127, PER.C6, YB2/0 or HEK293 could be used, for their ability to process to the right post-translational modifications of the mutated Annexin A5 polypeptides of the present invention. The construction of expression vectors in accordance with the invention, and the transformation of the host cells can be carried out using conventional molecular biology techniques. The mutated Annexin A5 polypeptides of the invention, can, for example, be obtained by culturing genetically transformed cells in accordance with the invention and recovering the mutated Annexin A5 polypeptide expressed by said cell, from the culture. They may then, if necessary, be purified by conventional procedures, known in themselves to those skilled in the art, for example by fractional precipitation, in particular ammonium sulfate precipitation, electrophoresis, gel filtration, affinity chromatography, etc. In particular, conventional methods for preparing and purifying recombinant proteins may be used for producing the proteins in accordance with the invention.

The mutated Annexin A5 polypeptides of the present invention and polynucleotides encoding thereof are typically used as medicament. In particular, the polynucleotides of the present invention (inserted or not into a vector) are particularly suitable for gene therapy.

A further object of the present invention thus relates to a method of therapy in a subject in need thereof comprising administering a therapeutically effective amount of a mutated Annexin A5 polypeptide of the present convention.

In particular, the mutated Annexin A5 polypeptides of the present invention are particularly suitable for the treatment of any diseases or conditions wherein intravascular hemolysis or hemolytic anemia occurs.

The mutated Annexin A5 polypeptides of the present invention are particularly suitable for the
(a) for prevention or reduction of risk of thrombosis (such as atherothrombosis) and/or plaque rupture, or for administration to patients belonging to a risk group, including but not limited to systemic lupus erythematosus (SLE) patients and/or patients who have or have had (or are at risk of) a upper respiratory tract or other infection (including pneumococcal infection) that can cause increased levels of antiphospholipid related antibodies, or to treat (either actively or prophylactically) or reduce the risk of thromboembolism, hemorrhagic or vasculitic stroke, myocardial infarction, angina pectoris or intermittent claudication, unstable angina, other forms of severe angina, or transient ischemic attacks (TIA)
(b) for the treatment, prophylaxis or reduction of risk of vascular dysfunction, angina pectoris, ischaemic heart disease, peripheral artery disease, systolic hypertension, migraine, type 2 diabetes and erectile dysfunction, reducing ischemic pain and/or treatment of a vascular disease rupture,
(c) for the prophylaxis or treatment of restenosis (in particular neointima formation or thickening), or vascular inflammation;
(d) for use in inhibiting the activity of oxidized cardiolipin (oxCL) and for treating, preventing and/or reducing the risk of developing a cardiovascular disease, an auto-immune disease or inflammatory condition, including but not limited to the following diseases: cardiovascular disease (CVD), diabetes II, Alzheimer's disease, dementia in general, rheumatic diseases, atherosclerosis, high blood pressure, acute and/or chronic inflammatory conditions, myocardial infarction, acute coronary syndrome, stroke, transient ischemic attack (TIA), claudiction, angina, type I diabetes, rheumatoid arthritis, psoriasis, psoriatic arthritis, ankylosing spondylitis, Reiter's Syndrome, systemic lupus erythematosus, dermatomyositis, Sjogren's syndrome, lupus erythematosus, multiple sclerosis, myasthenia gravis, asthma, encephalitis, inflammatory bowel disease, chronic obstructive pulmonary disease (COPD), arthritis including osteoarthritis, idiopathic inflammatory myopathies (IIM), dermatomyositis (DM), polymyositis (PM), inclusion body myositis, an allergic disorder and/or osteoarthritis in a mammal; and
(e) for the prevention and/or reduction of peri- or postoperative complications following surgical intervention, such as complications following vascular surgery, especially peripheral vascular surgery.

In some embodiments, the mutated Annexin A5 polypeptide of the present invention is used for the treatment of a hematological disorders, including but not limited to sickle cell disease. As used herein, the term “sickle cell disease” or “SCD” has its general meaning in the art and refers to a hereditary blood disorder in which red blood cells assume an abnormal, rigid, sickle shape. Sickling of erythrocytes increases cell rigidity and osmotic fragility, makes erythrocyte movement across capillaries difficult, damages the vascular bed, triggers inflammation, participates in multicellular vaso-occlusions, and creates episodes of focal ischemia in multiple tissues and organs. Combined, these multiple cellular and tissue modifications greatly increase the risk of various life-threatening complications. The term includes sickle cell anemia, hemoglobin SC disease and Hemoglobin sickle beta-thalessemia.

In some embodiments, the mutated Annexin A5 polypeptide of the present invention is particularly suitable for the treatment of vaso-occlusion. As used herein, the term “vaso-occlusion” has its general meaning in the art and refers to a common complication of sickle cell disease which leads to the occlusion of capillaries and the restriction of blood flow to an organ, resulting in ischaemia, with vascular dysfunction, tissue necrosis, and often organ damage. Vaso-occlusions are usually a constituent of vaso-occlusive crises, but they may also be more limited, clinically silent, and not cause hospitalization for vaso-occlusive crisis. As used herein, the term “vaso-occlusive crisis” has its general meaning in the art and refers to a common painful complication of sickle cell disease which leads to hospitalization, in association with occlusion of capillaries and restrict blood flow to an organ resulting in ischaemia, severe pain, necrosis, and most often with transient vaso-occlusions, and organ damage. In particular, mutated Annexin A5 polypeptide of the present invention is particularly suitable for reperfusing the capillaries.

In some embodiments, the mutated Annexin A5 polypeptide of the present invention is particularly suitable for the treatment of acute and chronic vascular inflammation, primary or secondary vasculitis including but not limited to vasculitis with autoimmune components, and/or drug induced vasculitis. Accordingly, the present invention also provides a prophylactic or therapeutic method of treating, preventing or reducing the risk of vasculitis, including Behget Disease, Cutaneous Vasculitis, Eosinophilic Granulomatosis with Polyangiitis (EGPA), Giant Cell Arteritis, Granulomatosis with Polyangiitis (GPA), Immunoglobulin A-Associated Vasculitis (IgAV), Microscopic Polyangiitis (MPA), Polyarteritis Nodosa (PAN), Polymyalgia Rheumatica, and Takayasu Arteritis. Polymyalgia Rheumatica may be of particular interest.

In some embodiments, the mutated Annexin A5 polypeptide of the present invention is particularly suitable for the treatment of an ischemic retinopathy. As used herein, the “ischemic retinopathy” has its general meaning in the art and refers to a group of diseases where progressive irreversible visual loss occurs as a consequence of retinal neovascularization. Examples of ischemic retinopathies include diabetic retinopathy, age-related macular degeneration, neovascular glaucoma, retinopathy of prematurity, sickle-cell retinopathy, retinal vein occlusion, oxygen induced retinopathy, and neovascularization due to ocular insults such as traumatic or surgical injury, or transplantation of eye tissue.

In some embodiments, the mutated Annexin A5 polypeptide of the present invention is particularly suitable for the treatment of a viral infection. Typically, the infection is caused by a virus selected from the group consisting of (a) a virus capable of causing hemorrhagic fever (VHF), and (b) a virus that presents phosphatidylserine (PS) and mediates cell infection and/or internalization through PS binding. The viral hemorrhagic (or haemorrhagic) fevers (VHFs) are a diverse group of animal and human illnesses that may be caused by at least five distinct families of RNA viruses: the families Arenaviridae, Filoviridae, Bunyaviridae, Flaviviridae, and Rhabdoviridae. All types of VHF may be characterized by fever and bleeding disorders and all can progress to high fever, shock and death in many cases. Signs and symptoms of VHIFs characteristically include fever and increased susceptibility to bleeding (bleeding diathesis). Manifestations of VHF often also include flushing of the face and chest, small red or purple spots (petechiae), frank bleeding, swelling caused by edema, low blood pressure (hypotension), and shock. Malaise, muscle pain (myalgia), headache, vomiting, and diarrhea occur frequently. The severity of symptoms varies with the type of virus, with the “VHF syndrome” (capillary leak, bleeding diathesis, and circulatory compromise leading to shock) appearing in a majority of patients with filovirus hemorrhagic fevers (e.g., Ebola and Marburg), CCHF, and the South American hemorrhagic fevers, but in a small minority of patients with dengue, RVF, and Lassa fever. In some embodiments, the VHF may be Ebola, and subject may display one or more symptoms of Ebola, such as symptoms selected from initial clinical symptoms, such as excessive or profuse sweating, the onset of fever, myalgia, general malaise, and/or chills; and/or flu-like symptoms optionally accompanied by gastro-intestinal symptoms; maculo-papulary rash, petichae, conjunctival hemorrhage, epistaxis, melena, hematemesis, shock and/or encephalopathy; leukopenia (for example, associated with increased lymphoid cell apoptosis), thrombocytopenia, increased levels of aminotransferase, thrombin and/or partial thromboplastin times, fibrin split products detectable in the blood, and/or disseminated intravascular coagulation (DIC).

In some embodiments, the mutated Annexin A5 polypeptide of the present invention is particularly suitable for the treatment of venous thromboembolism and arterial thrombosis in a patient suffering from a viral infection. In some embodiments, the patient suffers from an influenza infection. As used herein, the term “influenza infection” has its general meaning in the art and refers to the disease caused by an infection with an influenza virus. In some embodiments of the invention, influenza infection is associated with Influenza virus A or B. In some embodiments of the invention, influenza infection is associated with Influenza virus A. In some specific embodiments of the invention, influenza infection is cause by influenza virus A that is H1N1, H2N2, H3N2 or H5N1. In some embodiments, the patients suffer from a coronavirus infection. As used herein, the term “coronavirus” has its general meaning in the art and refers to any member of members of the Coronaviridae family. Coronavirus is a virus whose genome is plus-stranded RNA of about 27 kb to about 33 kb in length depending on the particular virus. The virion RNA has a cap at the 5′ end and a poly A tail at the 3′ end. The length of the RNA makes coronaviruses the largest of the RNA virus genomes. In particular, coronavirus RNAs encode: (1) an RNA-dependent RNA polymerase; (2) N-protein; (3) three envelope glycoproteins; plus (4) three non-structural proteins. These coronaviruses infect a variety of mammals and birds. They cause respiratory infections (common), enteric infections (mostly in infants >12 mo.), and possibly neurological syndromes. Coronaviruses are transmitted by aerosols of respiratory secretions. In some embodiments, the patient suffers from a SARS-CoV-2 infection. As used herein, the term “Severe Acute Respiratory Syndrome coronavirus 2” or “SARS-CoV-2” has its general meaning in the art and refers to the strain of coronavirus that causes coronavirus disease 2019 (COVID-19), a respiratory syndrome that manifests a clinical pathology resembling mild upper respiratory tract disease (common cold-like symptoms) and occasionally severe lower respiratory tract illness and extra-pulmonary manifestations leading to multi-organ failure and death. In particular, the term refers to the severe acute respiratory syndrome coronavirus 2 isolate 2019-nCoV_HKU-SZ-005b_2020 for which the complete genome is accessible under the NCBI access number MN975262. In some embodiments, the patient suffers from Covid-19. As used herein, the term “Covid-19” refers to the respiratory disease induced by the Severe Acute Respiratory Syndrome coronavirus 2.

It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

According to the invention, the mutated Annexin A5 polypeptide or the nucleic acid molecule (inserted or not into a vector) of the present invention is administered to the subject in the form of a pharmaceutical composition. Typically, the mutated Annexin A5 polypeptide or the nucleic acid molecule (inserted or not into a vector) of the present invention may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The mutated Annexin A5 polypeptide or the nucleic acid molecule (inserted or not into a vector) of the present invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1: Polypeptide sequences of Annexin-A5 and Annexin-A5 mutants

FIG. 2: Sickle Cell Disease patients with active intravascular hemolysis.

We identified Sickle Cell Disease patients (homozygous HbS, without ongoing hydroxyurea (HU) treatment) undergoing active intravascular hemolysis, by measuring heme-related absorbance at 415 nm in their PFP. We selected PFP samples that displayed Abs415 increased at least two-fold compared to healthy control levels. This accounted for about 20% of plasma collected in our collection of 200+ patients in our declared biological collections. PFP with elevated Abs415 were classified as ‘active intravascular hemolysis’ (SCD hemol.) and retained in priority for inhibition studies with Annexin-A5 polypeptides.

FIG. 3: Thrombin generation time in plasma from healthy volunteers and Sickle Cell Disease patients

Thrombin generation kinetic curves were generated in recalcified PFP from healthy volunteers (Blood donors of EFS; Left panels), or from Sickle Cell Disease patients (homozygous HbS, not treated with hydroxyurea; Right panels) displaying active intravascular hemolysis (elevated heme-related absorbance at 415 nm). Thrombin generation kinetic curves were produced in the absence (vehicle, PBS), or presence of wild type recombinant annexin-A5 polypeptides (rhA5-WT), or three recombinant annexin-A5 mutant polypeptides (rhA5-E, rhA5-R and rh-A5-Y) at 30 nM.

FIG. 4: Thrombin generation time in plasma from Sickle Cell Disease patient with ongoing hydroxyurea (HU) treatment.

Thrombin generation kinetic curves were generated in recalcified PFP from Sickle Cell Disease patients (homozygous HbS) under ongoing hydroxyurea (HU) therapy, which now represent a significant proportion of Sickle Cell Disease patients in many Western countries. Thrombin generation kinetic curves were produced in the absence (vehicle, PBS), or presence of wild type recombinant annexin-A5 polypeptides (rhA5-WT), or three recombinant annexin-A5 mutant polypeptides (rhA5-E, rhA5-R and rh-A5-Y) at 30 nM.

FIG. 5: Thrombin generation time in plasma from hospitalized COVID-19 patients.

Thrombin generation kinetic curves were generated in recalcified PFP from hospitalized COVID-19 patients. Severe COVID-19 patients were included after hospitalization for respiratory difficulties, on day 2-3 after admission, after they have received oxygenotherapy (no mechanical ventilation) plus prophylactic anticoagulation treatment with Low Molecular Weight Heparins (all but one at high prophylactic doses). Thrombin generation kinetic curves were produced in the absence (vehicle, PBS), or presence of wild type recombinant annexin-A5 polypeptides (rhA5-WT), or three recombinant annexin-A5 mutant polypeptides (rhA5-E, rhA5-R and rh-A5-Y) at 30 nM.

FIG. 6: Vaso-occlusive events in transgenic mice with Sickle Cell Disease.

Hypoxia/reoxygenation in HbSAD transgenic mice produced a major vaso-occlusive episode (H/R baseline), with a drop in cardiac output observed by echo-Doppler in the pulmonary artery compared with normoxic values (Normoxia) (Top panel). In contrast, heartrate remained unaffected (Bottom panel). This combination of features translated a drastic increase in vasuloresistance in the lung, and dispersed vaso-occlusions throughout the pulmonary capillary bed. After the vaso-occlusive episode was validated, the intravenous injection of recombinant annexin-A5 mutant polypeptides (rhA5-E, rhA5-R; 2.5 μg/mouse) were found to accelerate pulmonary reperfusion significantly (p<0.05 at 15 and 20 minutes after injection; n=4-6) compared to vehicle alone (PBS). A near-normal cardiac output was restored within 15 minutes after injection of annexin-A5 mutants, whereas spontaneous pulmonary reperfusion (vehicle, PBS) only occurs in about 4 to 8 hours after H/R. This suggested that recombinant mutant Annexin-A5 polypeptides acted to release vaso-occlusions and facilitated stressed erythrocyte circulation through the capillary bed, and can ameliorate tissue perfusion in ‘curative’ therapeutic applications for pre-established vascular occlusions.

### EXAMPLE

Methods

Collection of Blood Samples

Venous blood samples were collected into Monovette® (Sarstedt, Nümbrecht, Germany) syringes containing 1/10 volume of 0.10⁶ M sodium citrate. All analyses were performed within two hours after blood collection. Platelet-Rich Plasma (PRP) was prepared by blood centrifugation at 190 g for 10 min at 20° C. The PRP was recovered and platelet-free plasma (PFP) was obtained by double centrifugation of the PRP at 2500 g for 15 min at 20° C. PFP was immediately frozen at −80° C. and thawed 15 min at 37° C. when needed. PFP was generated from different types of patients, including healthy volunteers (Blood donors), Sickle Cell Disease patients (homozygous HbS) without or with ongoing hydroxyurea (HU) treatment, as well as hospitalized COVID-19 patients (on day 2-3 after admission, and after they have received prophylactic anticoagulation treatment with Low Molecular Weight Heparins at high dose, plus oxygenotherapy).

Thrombin Generation Assays

Calibrated automated thrombogram (CAT) was performed at 37° C. in a microtiter plate fluorometer (Fluoroskan Ascent, ThermoLabsystems, Helsinki, Finland) using a dedicated software program (Thrombinoscope BV, Maastricht, The Netherlands). Coagulation cascade activation was triggered by recalcification of the samples only, without any exogenous phospholipid of tissue factor addition. This allowed the evaluation of Annexin-A5 polypeptides for the inhibition of PS carried by endogenous physiopathological membranes (such as extracellular vesicles) in their native state and in their complex multifactorial environment (including hemolysis products), as they exist in the plasma of each type of patient. Thrombin generation curves were recorded in triplicate using PFP in the absence or in the presence or absence of recombinant Annexin-A5 (WT) and Annexin-A5 mutants at several final concentrations (0.3, 3, 30, 300 nM). Thrombin generation was presented as kinetic curves of thrombin activity, over up to 90 minutes. Four main types of data can be extracted from the major components of the reaction curves, including the total amount of thrombin activity (i.e. the endogenous thrombin potential (ETP, assessed as the area under the curve), as well as the maximal thrombin generation rate, the lag time, the time-to-peak). The concentration of wild type Annexin-A5 (rhAnn-A5-WT, or rhA5-WT), or the concentration of Annexin-A5 mutant that produced a 50% inhibition of ETP was defined as IC₅₀ for that polypeptide.

Murine Model of Sickle Cell Disease and Vaso-Occlusive Episodes.

We used 10-18 week old HbSAD transgenic male mice (expressing SAD-mutated hemoglobin chains) and induced vaso-occlusive events akin to vaso-occlusive crises in SCD patients. HbSAD mice are a validated model for inducible VOC, particularly suited to the study of RBC degradation products and their inhibitors. HbSAD mice, like SCD patients, also present neutrophil hyperleukocytosis.

Hemorrheological parameters were first collected at rest (during normoxia) by echo-Doppler. We characterized heart rate, and cardiac output in the pulmonary artery. Vaso-occlusive episodes were then triggered by Hypoxia/reoxygenation (H/R), by placing the mice in 9% 02 overnight (12-16 hours), followed by sudden reoxygenation (room air) for 30 minutes. Heart rate and cardiac output in the pulmonary artery were collected again by echo-Doppler (H/R baseline measurement). Mice were considered to go through a major vaso-occlusive episode when hypoxia/reoxygenation produced a sudden drop in cardiac output, whilst heartrate remained unaffected. This combination of phenomena translated a drastic increase in vasuloresistance in the lung, a difficulty for blood to traverse the pulmonary vasculature due to dispersed vaso-occlusions throughout the distal capillary bed.

After the vaso-occlusive episode has been characterized, recombinant Annexin-A5 polypeptides were injected intravenously. Heart rate and cardiac output in the pulmonary artery were collected again by echo-Doppler over an observation period of about 45 minutes after injections. Pulmonary reperfusion was considered to be achieved when cardiac output (as observed in the pulmonary artery) had returned to normoxia levels (pre-H/R). Full spontaneous pulmonary reperfusion is known to occur in about 4 to 8 hours after H/R.

Results

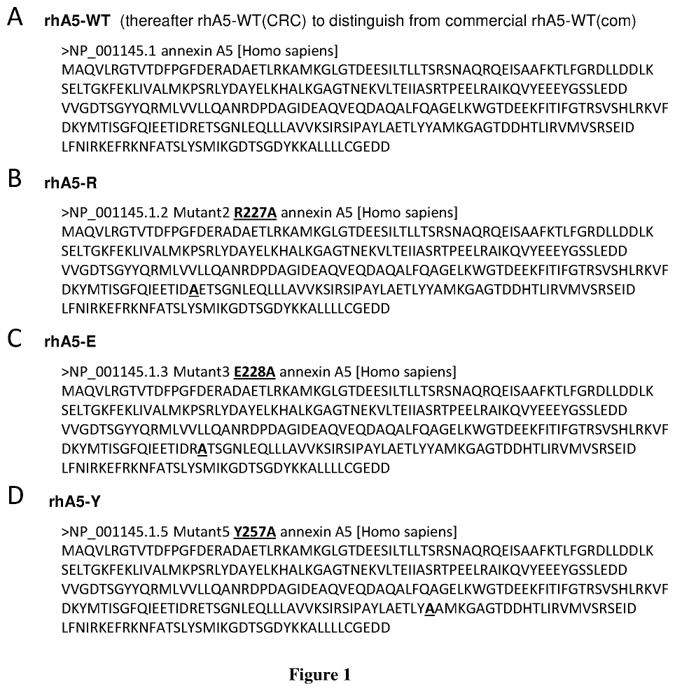

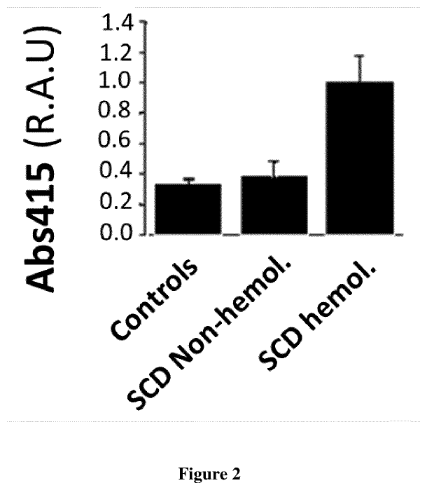

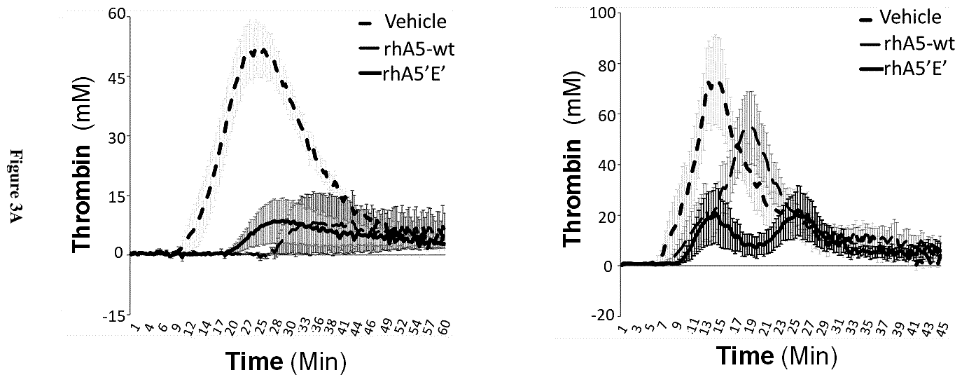

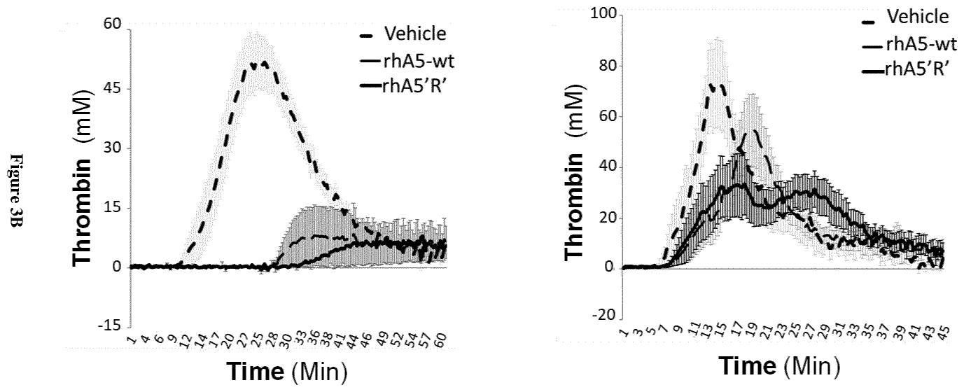

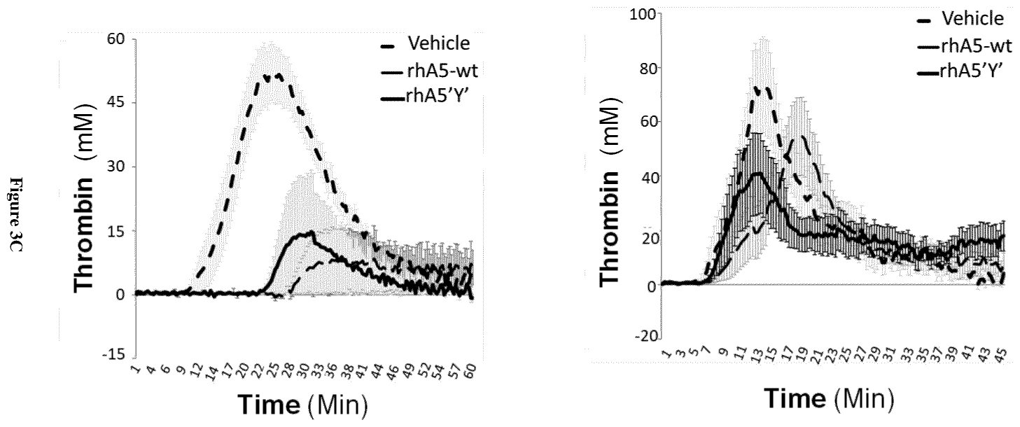

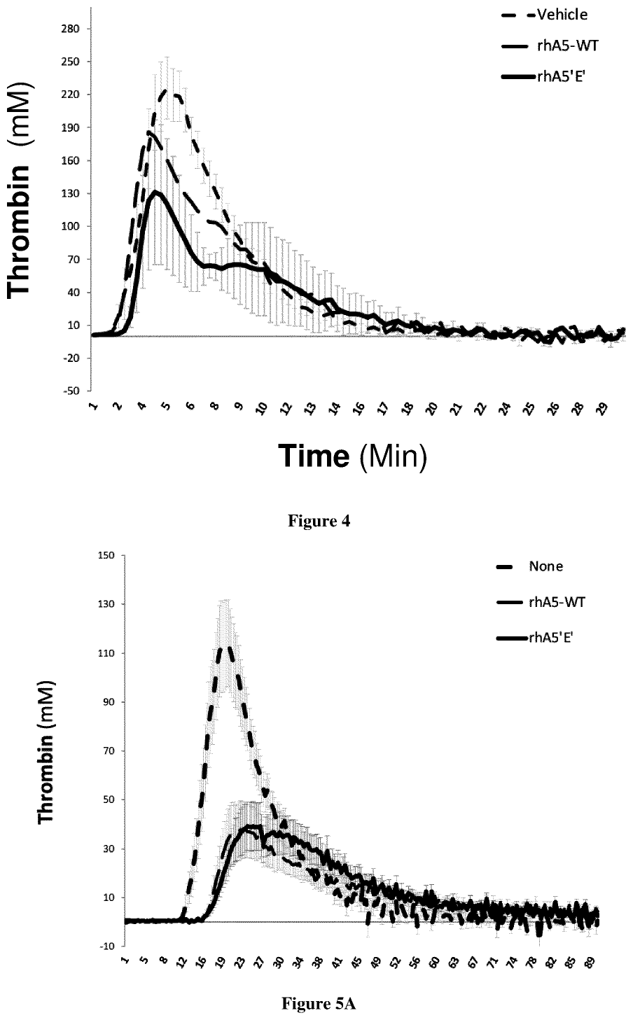

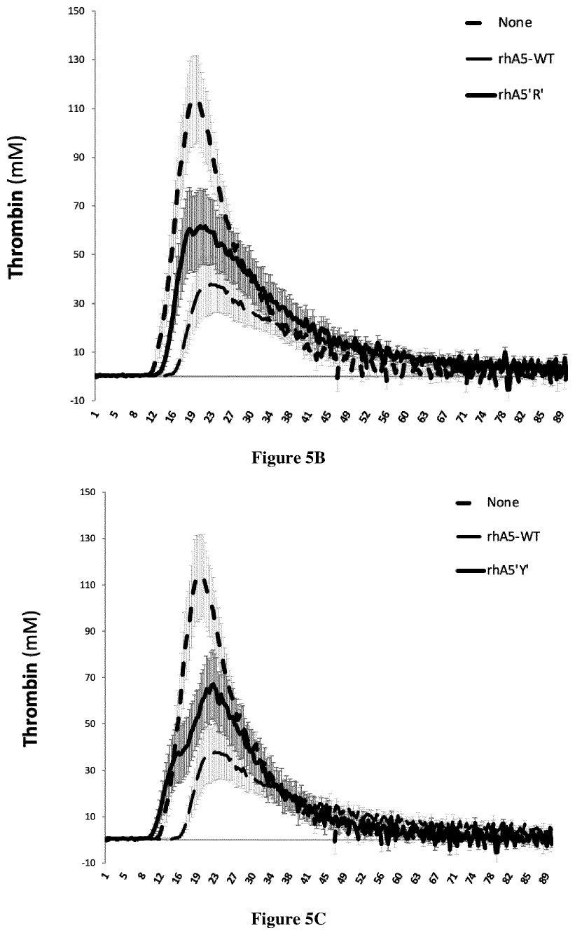

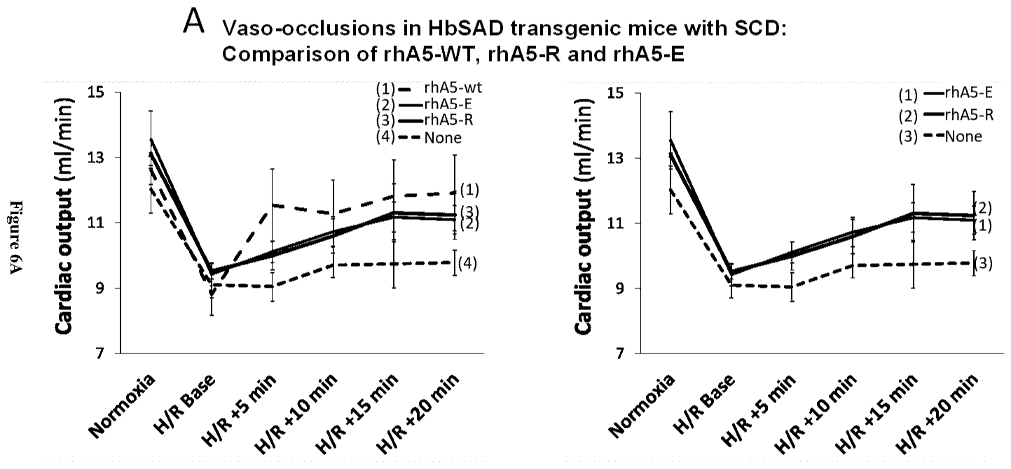

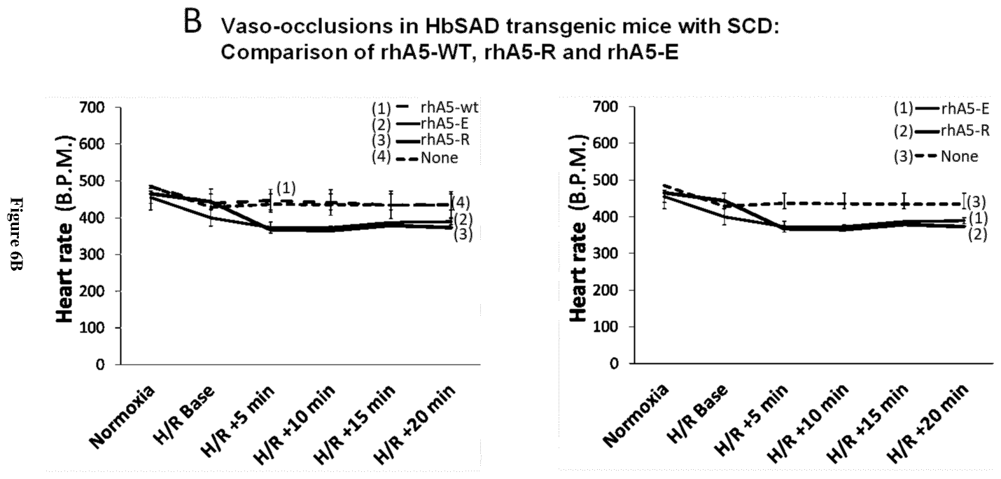

We generated recombinant human mutated annexin-A5 polypeptides having two characteristics: low Heme-interaction, and high TGT (thrombin generation time)-inhibition (FIG. 1). We next evaluated the functionality of said Annexin A5 polypeptides in SCD patients. We can divide the patients in 2 groups, demonstrating active ongoing intravascular hemolysis, and those who do not at the time of blood sampling. This can be determined by an increased absorbance at 415 nm (Soret band) wherein a threshold above 0.5 arbitrary units in plasma to distinguish non-hemolytic patients from patients with ongoing intravascular hemolysis (FIG. 2). SCD patients with active intravascular hemolysis displayed accelerated and increased thrombin generation in plasma versus non-hemolytic patients, as demonstrated by the main data derived from TGT curves (not shown). We showed that the mutated A5 polypeptides are effective for reducing thrombin generation in particular in the haemolytic SCD patients (FIG. 3). SCD patients treated with hydroxyurea demonstrated an accelerated and increased thrombin generation in plasma versus healthy subjects, despite hydroxyurea treatment, as demonstrated by TGT curves (not shown). We showed that rhA5-‘E’ mutant and rhA5-WT displayed a significant and specific ability to reduce thrombin generation in these plasmas, beyond the benefits brought by hydroxyurea (FIG. 4). We then showed that rhA5-‘E’ (i.e A5 polypeptide comprising E228A mutation), rhA5-‘R’ (A5 polypeptide comprising R227A mutation) and rhA5-‘Y’ mutants (A5 polypeptide comprising Y257A mutation), like rhA5-WT displayed a significant and specific ability to reduce thrombin generation in plasmas of hospitalized COVID-19 patients beyond the benefits of prophylactic anticoagulation (FIG. 5). Finally, in a murine model of sickle cell disease with vaso-occlusive episodes, we showed that rhA5-‘E’ (E228A) and rhA5-‘R’ (R227A) mutants, like rhA5-WT accelerated lung reperfusion after pre-established vaso-occlusive events, compared to vehicle (PBS) (FIG. 6).

### REFERENCES

Throughout this application, various references describe the state of the art to which this invention pertains. The disclosures of these references are hereby incorporated by reference into the present disclosure.

1. A mutated Annexin A5 polypeptide that comprises an amino acid sequence having at least 90% identity with the amino acid sequence as set forth in SEQ ID NO: 1 wherein at least one amino acid at position 227, 228 or 257 is mutated.

2. The mutated Annexin A5 polypeptide of claim 1 wherein the amino acid at position 227, 228 or 257 is substituted.

3. The mutated Annexin A5 polypeptide of claim 1 wherein the amino acid (R) at position 227 is substituted by the amino acid (A).

4. The mutated Annexin A5 polypeptide of claim 1 wherein the amino acid (E) at position 228 is substituted by the amino acid (A).

5. The mutated Annexin A5 polypeptide of claim 1 wherein the amino acid (Y) at position 257 is substituted by the amino acid (A).

6. The mutated Annexin A5 polypeptide of claim 1 that comprises an amino acid sequence having at least 90% identity with the amino acid sequence as set forth in SEQ ID NO: 1 that comprises at least one mutation selected from the group consisting of R227A, E228A and Y257A.

7. The mutated Annexin A5 polypeptide of claim 1 that comprises an amino acid sequence having at least 90% identity with the amino acid sequence as set forth in SEQ ID NO: 2, 3 or 4.

8. The mutated Annexin A5 polypeptide of claim 1 that comprises the amino acid sequence as set forth in SEQ ID NO:3.

9. A polynucleotide that encodes for the mutated Annexin A5 polypeptide of claim 1.

10. A vector that comprises the polynucleotide of claim 9.

11. A host cell which is transformed with the polynucleotide of claim 9 or a vector comprising the polynucleotide.

12. A method of therapy in a subject in need thereof comprising administering to the subject a therapeutically effective amount of the mutated Annexin A5 polypeptide of claim 1.

13. The method of claim 12 wherein the subject suffers from a disease or condition wherein intravascular hemolysis or hemolytic anemia occurs.

14. The method of claim 12 wherein the method prevents or reduces a risk of thrombosis in the subject.

15. The method of claim 12 wherein the subject suffers from sickle cell disease and administration of the mutated Annexin A5 polypeptide prevents or treats vaso-occlusions in the subject.

16. The method of claim 12 wherein the subject suffers from a viral infection.

17. A pharmaceutical composition that comprises an amount of the mutated Annexin A5 polypeptide of claim 1.

18. The method of claim 16, wherein the viral infection is a SAR-CoV-2 infection.
